(19) United States
(12) Patent Application Publication
(10) Pub. No.: US 2009/0311798 A1
(43) Pub. Date: Dec. 17, 2009

(54) SE(R)RS DISPLACEMENT ASSAY

(75) Inventors: Reinhold Wimberger-Friedl, Eindhoven (NL); Gardiye Pattinige Kusalini Chamindie Punyadeera, Eindhoven (NL); Kristiane Anne Schmidt, Eindhoven (NL); René Van Den Ham, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/300,493

(22) PCT Filed: May 9, 2007
(86) PCT No.: PCT/IB2007/051737

§ 371 Date: Nov. 12, 2008

(30) Foreign Application Priority Data
May 16, 2006 (EP) 06114018.2
Aug. 11, 2006 (EP) 06118777.9

(51) Int. Cl. G01N 21/65; C07H 21/00; G01N 21/01; G01N 21/64

(52) U.S. Cl. 436/171; 536/24.3; 422/68.1; 422/82.05; 422/82.08

(57) ABSTRACT

A detection method is described for detecting an analyte, in particular nucleic acids, in a sample using SE(R)RS, without the need for labeling of the analyte. The detection method is based on the displacement of a labeled surrogate target probe from a capture probe by the analyte in the sample. The present invention also provides a corresponding detection system, to a disposable cartridge for use in such a system, and to a combination of surrogate target probe and capture probe.

### FIELD OF THE INVENTION

The present invention relates to a competitive SE(R)RS assay method to detect analytes, in particular nucleic acids in samples, and to tools and devices for performing these methods.

### BACKGROUND OF THE INVENTION

Sensitive, specific and fast detection of the presence of certain biomolecules in a sample is of great importance for molecular diagnostics applications. Typically, so-called capture probes are immobilized on a sensor surface and the specific binding of the target to the capture probes is monitored either by detection of a label introduced into the target or by a second probe provided with a label. This requires the transport of the target, probes and labels to the surface. Since diffusion of macromolecules is relatively slow the binding to the surface becomes the time-critical step in the analysis. The binding of the target to the surface can be reversible, e.g., in DNA hybridizations the hybridized target molecules may be stripped or melted off the surface and the substrate can be re-used after washing. However, this is time-consuming. Therefore so-called homogeneous assays, whereby the analysis is carried out in solution, are advantageous. In effect, diffusion distances are very small and devices can be reused easily. Nevertheless, working with immobilized capture probes has the advantage of the ease of multiplexing (i.e. determining different targets at the same time) by a pattern-wise deposition.

Recently a technique has been introduced which allows multiplexed, sensitive measurements in homogeneous solution, the so-called surface-enhanced (resonant) Raman spectroscopy (SE(R)RS). For SE(R)RS measurements, the molecules to be detected need to adsorb on a nanostructured noble metal surface. The Raman signal is enhanced by several orders of magnitude due to interaction between the optical field of the excitation beam and the plasmonic field at the metal surface (SERS) and also due to resonant excitation of the chromophore (SERRS). SE(R)RS has the unique feature that the scattered light consists of sharp, molecule-specific vibrational bands which makes discrimination of multiple analytes within one solution possible. In the application of SERRS to DNA detection, a SERRS label with a dual functionality is incorporated into the nucleotide sequence of interest, e.g. by PCR. The SERRS label will attach to a silver surface by electrically (positive) charged groups and generate the Raman signal by dye moieties. The Raman signal is strongly amplified by the plasmonic field created at the surface of the confined silver particle, allowing for extremely sensitive detection (in the picomolar range).

Nevertheless, the above-described systems have been observed to have several limitations. Detection of DNA, for example, has been only feasible when the label is incorporated into the DNA, which generally requires an amplification step. Moreover, the SERRS spectrum of the dye labels does not depend on the sequence or structure of the nucleic acid attached to it, meaning that unincorporated primers or unhybridized probes, respectively, need to be separated from the solution before the measurement.

U.S. Pat. No. 6,750,065 describes the detection of proteins using SE(R)RS, whereby an antigen is detected based on its capacity to displace a SE(R)RS-labeled analyte-like compound from an analyte-specific antibody. Upon displacement, the SE(R)RS label bound to the analyte-like compound is detected by adsorption of a SE(R)RS surface thereto.

### BRIEF SUMMARY OF THE INVENTION

It is an object of the present invention to simplify the in vitro detection of nucleic acids in samples by using an alternative SE(R)RS assay in which labeling of the analyte is not required.

The above objective is accomplished by providing methods for competitive SE(R)RS according to the present invention and to tools and devices for use in these methods.

It is an advantage of the present invention that multiplexing is easily feasible because the sharp molecule-specific vibrational bands of SE(R)RS spectra enable sensitive and accurate detection of multiple labels simultaneously.

It is furthermore an advantage of the present invention that SE(R)RS and fluorescence can be measured simultaneously providing an extra validation of the method.

Another advantage of the present invention is that real-time measurement of either or both SE(R)RS and fluorescence can provide fast answers as to whether or not a certain analyte is present.

Moreover, the occurrence of a fluorescent signal readily points to the presence of analyte in the sample. A rapid, quantitative test can thus be provided through detection of fluorescence only.

It is also an advantage of the present invention that an internal standard can be provided enabling corrections for variations e.g. in optical detection, in aggregation of silver nanoparticles, etc.

It is also an advantage of the present invention that the label is brought near the SE(R)RS surface without the need of direct adsorption of the label to the surface. Instead, the label is brought in the proximity of the surface via binding of the surrogate target probe (comprising the label) to the capture probe through oligonucleotide hybridization, a process which can be readily controlled. In this way, the lack of control of the surface adsorption process of different labels onto the SE(R)RS surface which is a problem in current SE(R)RS based detection methods, is circumvented.

Particular and preferred aspects of the invention are set out in the accompanying independent and dependent claims. Features from the dependent claims may be combined with features of the independent claims and with features of other dependent claims as appropriate and not merely as explicitly set out in the claims.

In a first aspect of the present invention, the presence and/or quantity of at least one analyte in a sample is determined by a displacement assay based on the steps of (a) contacting the sample with at least one target-specific capture probe and at least one displaceable surrogate target probe (which is labeled), whereby the target-specific capture probe(s) is/are either covalently bound to a SE(R)RS surface or step (a) further comprises contacting the target-specific capture probe(s) with a SE(R)RS surface, and (b) detecting the signal generated by the label(s) in the surrogate hybrid(s) formed by the binding of the displaceable surrogate target probe(s) and the target-specific capture probe(s), which signal(s) is/are proportionate to the presence and/or quantity of the analyte(s) in the sample.

Within this method, step (a) may further comprise the steps of (I) contacting the target-specific capture probe(s) with the displaceable surrogate target probe(s) so as to obtain the surrogate hybrid(s), whereby the target-specific capture probe(s) is/are either covalently bound to a SE(R)RS surface and the surface-adsorbed surrogate hybrid(s) is/are so obtained or step (I) further comprises contacting the target-specific capture probe(s) with a SE(R)RS surface or said step (I) further comprises contacting the surrogate hybrid(s) with a SE(R)RS surface such that the surrogate hybrid(s) become(s) adsorbed on the SE(R)RS surface as surface-adsorbed surrogate hybrid(s), and (II) contacting the surface-adsorbed surrogate hybrid(s) with the sample so as to allow displacement of the displaceable surrogate target probe(s) by the analyte(s).

Within this method, step (b) may further comprise the steps of (III) detecting the signal(s) of the label(s) in the surface-adsorbed surrogate hybrid(s) after step (I) using either or both SE(R)RS and fluorescence; and (IV) detecting the signal(s) of the surface-adsorbed surrogate hybrid(s) after step (II) using the same detection method or methods used in step (III). The difference in the signal(s) obtained in steps (III) and (IV) is taken as proportionate to the presence and/or quantity of the analyte(s) in the sample.

Step (a) in the above described method may also further comprise the steps of (V) contacting the target-specific capture probe(s) with the displaceable surrogate target probe(s) so as to obtain the surrogate hybrid(s), (VI) contacting the surrogate hybrid(s) with the SE(R)RS surface such that the surrogate hybrid(s) become(s) adsorbed on the surface forming the surface-adsorbed surrogate hybrid(s), and (VII) contacting the surface-adsorbed surrogate hybrid(s) with the sample so as to allow displacement of the displaceable surrogate target probe(s) by the analyte(s).

To enable calibration or correction for variations e.g. aggregate or optical variations, a standard is included in the above described method as provided by the steps (c) contacting a standard capture probe and a standard probe (which is labeled) thereby obtaining a standard hybrid, and contacting the standard hybrid with a SE(R)RS surface thereby obtaining a surface-adsorbed standard hybrid, and (d) detecting the signal generated by the label in said surface-adsorbed standard hybrid.

In a second aspect of the present invention a combination of surrogate target probe and capture probe is disclosed whereby the oligonucleotide of the surrogate target probe is characterized by a melting temperature that is lower than the melting temperature of an oligonucleotide that is 100% complementary to the capture probe's oligonucleotide, and the label attached to the surrogate target probe has either or both surface-enhanced (resonance) Raman scattering (SE(R)RS) and fluorescence activity.

In a third aspect of the present invention, a disposable cartridge for use in a system for detecting the presence or amount of at least one analyte in a sample is disclosed and comprises (a) a set of sources comprising source of sample, at least one source of surrogate target, at least one source of target-specific capture probe, and at least one source of additives serving in the detection, (b) means for contacting specified volumes from the sources, and (c) means for ensuring the provision of the fluids from the sources to the contacting means. This cartridge may further comprise at least one source of internal standard reagents, and a window for detecting the signal(s) generated at said contacting means.

In a fourth aspect of the present invention, a system for detecting the presence or amount of at least one analyte in a sample is disclosed and comprises (a) means for contacting the sample with at least one target-specific capture probe and at least one displaceable surrogate target probe (which is labeled) and (b) means for detecting the signal(s) generated by the label(s) in the surrogate hybrid(s) formed by the binding of the displaceable surrogate target probe(s) and the target-specific capture probe(s). This system may further comprise a means for calculating the amount of analyte(s) by comparing the detection signals detected in the contacting means at different time points in the provision of reagents from the sources to the contacting means.

The teachings of the present invention permit the design of improved methods and apparatus for the detection of analytes in samples.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic representation of an embodiment of the SERRS displacement assay. A: The capture probe, comprising a sequence complementary to a target sequence in the analyte, is adsorbed onto the surface of a citrate-reduced silver nanoparticle by electrochemical interaction between the negatively charged surface and the positively charged surface-seeking group. The displaceable surrogate target probe is complementary to the capture probe and therefore hybridizes to it. In this way, the SERRS dye comes close to the silver surface and a strong SERRS signal can be detected. B: After addition of a sample, the analyte comprising the target sequence competes with the surrogate target probe for hybridization to the capture probe. Since the displaceable surrogate target probe (comprising one or more SNPs) is not perfectly complementary to the capture probe, hybridization of the analyte is more efficient and the displaceable surrogate target probe is displaced from the silver surface by the analyte. The SERRS signal will thus diminish proportionately to the amount of analyte in the sample.

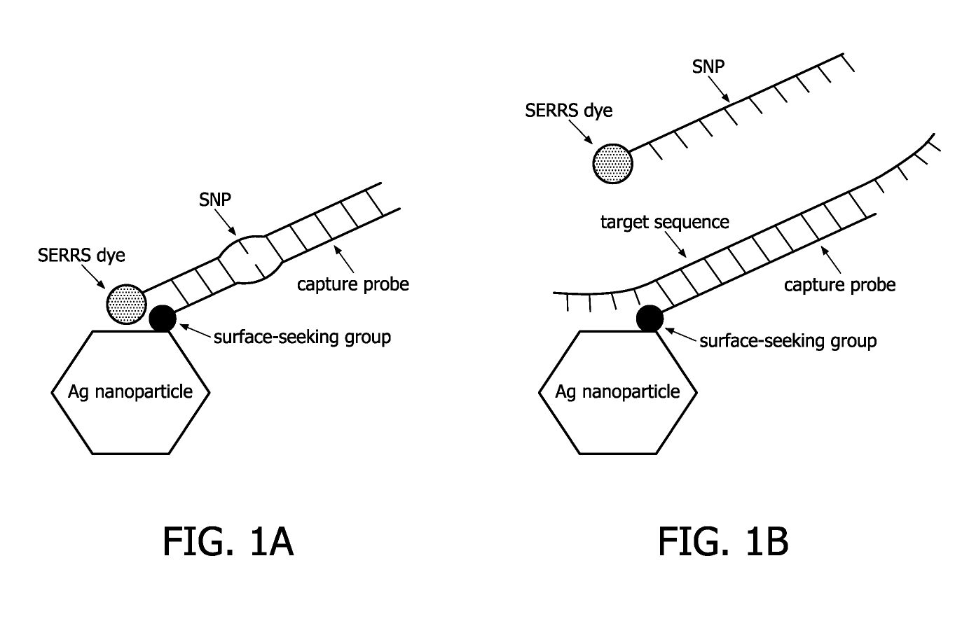

FIG. 2 is a graph showing the absorption spectrum of the SERRS dye HEX as a function of wavelength.

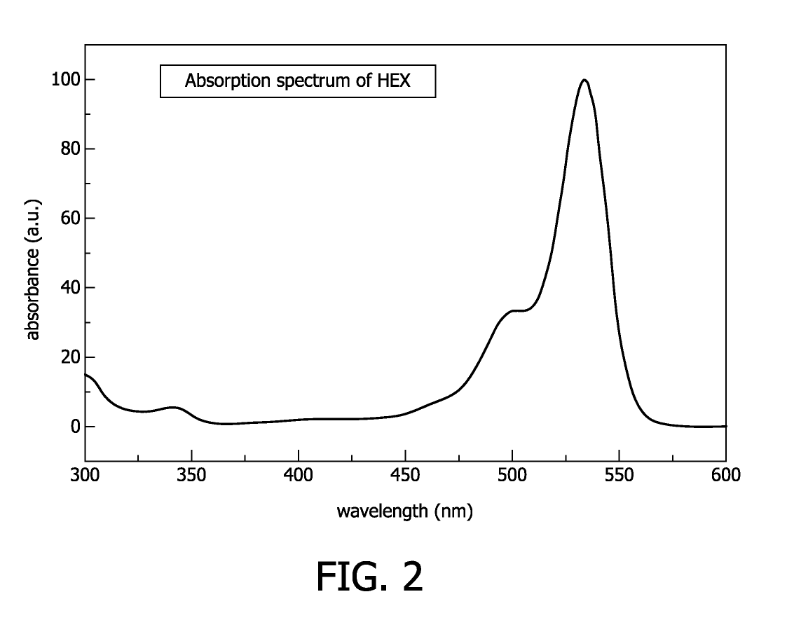

FIG. 3 is a graph showing the SERRS spectrum of a 5′ HEX labeled oligonucleotide containing aminopropargyl moieties to promote adsorption onto the silver surface. The SERRS signal (without background correction) was measured after adsorption of the oligonucelotide to spermine-aggregated silver nanoparticles.

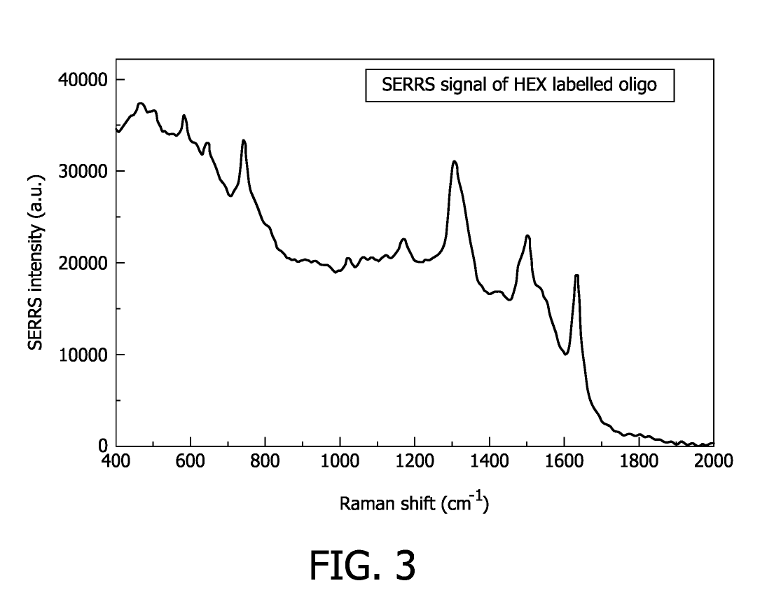

FIG. 4 is a block diagram illustrating a detection method according to one embodiment of the invention.

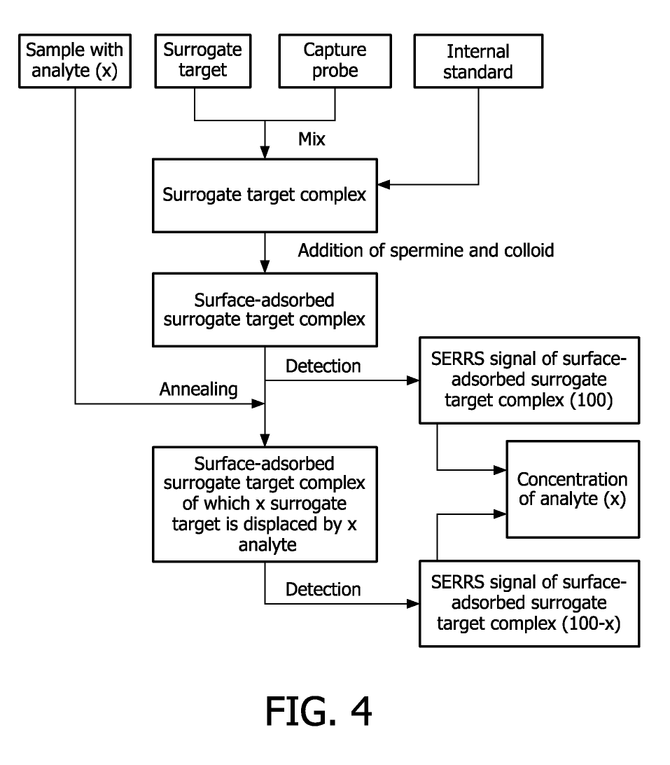

FIG. 5 is a schematic representation of a device according to one embodiment of the present invention.

In the different figures, the same reference signs refer to the same or analogous elements.

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. These definitions should not be construed to have a scope less than understood by a person of ordinary skill in the art.

### Definitions

An “analyte” as used herein refers to the substance to be detected and/or quantified by the methods of the present invention.

A “target sequence” as used herein refers to a specific nucleotide sequence within the analyte, used to specifically detect the analyte.

A “sample” as used herein relates to a composition believed to comprise at least one analyte of interest.

A “control sample” as used herein relates to a composition that is identical or very similar to the sample.

A “label” as used herein refers to a molecule or material capable of generating a detectable SE(R)RS or fluorescence signal.

An “oligonucleotide” as used herein refers to a sequence of between 5 and 200 nucleotides which can be either naturally occurring DNA or RNA nucleotides, modified or synthetic nucleotides or nucleotide analogs, the nucleotides being linked as DNA, RNA, PNA, or any backbone derivatized variation thereof.

A “capture probe” as used herein refers to an oligonucleotide sequence comprising a surface-seeking group which allows the binding of the capture probe to the SE(R)RS surface. The oligonucleotide sequence of a capture probe can be complementary to the target sequence (“target-specific capture probe”) and capable of specifically hybridizing to the target sequence or can be complementary to a different predetermined standard sequence (“standard capture probe”) which is part of the standard probe.

A “surrogate target probe” as used herein refers to a synthetic, labeled oligonucleotide, capable of specifically binding a capture probe. A “displaceable surrogate target probe” is a surrogate target probe which binds to the capture probe with a strength that is lower than the binding strength of a sequence which is unmodified and 100% complementary to the capture probe. In a “non-displaceable surrogate target probe” the sequence is completely complementary to that of a capture probe, either to a target-specific capture probe or to a standard capture probe (“standard probe”), resulting in the strong binding of the non-displaceable surrogate target probe to the corresponding capture probe. The label is attached to the surrogate target probe, e.g. by covalent linking, or it can be a separate entity which binds to the surrogate target probe.

A “surrogate hybrid” as used herein refers to the combination of a surrogate target probe and a capture probe.

A “target hybrid” as used herein refers to the combination of the target sequence within the analyte and a target-specific capture probe.

A “standard hybrid” as used herein, refers to a combination of a standard probe and a standard capture probe, which is either covalently or non-covalently linked to a SE(R)RS surface.

A “SE(R)RS surface” as used herein refers to a material which is capable of enhancing the signal of a SE(R)RS label.

A “surface-adsorbed surrogate hybrid”, a “surface-adsorbed target hybrid”, and a “surface-adsorbed standard hybrid” as used herein, refer to a surrogate hybrid, a target hybrid, and a standard hybrid, respectively that are adsorbed onto a SE(R)RS surface.

The present invention provides methods, tools and devices for the detection of the presence and/or quantification of one or more analytes in a sample.

The invention is based on the observation that by detecting the analyte in a sample based on the displacement of a labeled probe, a one-step analyte-specific method is provided which does not require labeling of the analyte. This is an important time-saving improvement which further contributes to the reliability of detection, as it can be performed with reagents which are all standard and prefabricated.

The methods, tools and devices of the invention are based on the use of a displaceable surrogate target probe which is labeled and can hybridize to a target-specific capture probe which can bind to a SE(R)RS surface. Where the label of the displaceable surrogate target probe is a SE(R)RS label, hybridization to the capture probe and contacting with the SE(R)RS surface will result in the generation of a “surface-adsorbed surrogate hybrid” which enhances detection of the label. The presence of at least one mismatch (non-complementary nucleotide) to the sequence of the capture probe in the sequence of the displaceable surrogate target probe causes the binding of the displaceable surrogate target probe to the capture probe to be weak. Upon contacting the surrogate target probe bound to the capture probe (either as such or as a surface-adsorbed surrogate hybrid) with an analyte comprising a target sequence which is completely complementary to the capture probe, the displaceable surrogate target probe is displaced.

The analyte envisaged to be detected using the methods of the present invention is a nucleic acid. More particularly, the analyte is a DNA such as a gene, viral DNA, bacterial DNA, fungal DNA, mammalian DNA, plasmid DNA, or a DNA fragment. The analyte can also be RNA such as viral RNA, mRNA, rRNA. The analyte can also be cDNA, oligonucleotides, or synthetic DNA, RNA, PNA, LNA, a nucleotide sequence comprising one or more synthetic oligonucleotides, modified oligonucleotides or other nucleic acid analogs. The analyte may be nucleic acid originating from an organism such as but not limited to a virus, a bacterium, a microorganism such as Salmonella, Streptococcus, Legionella, E. coli, Giardia, Cryptosporidium, Rickettsia, a spore, a mold, a yeast, an algae, an amoebae, a dinoflagellate, a unicellular organism, a pathogen or a cell of a multicellular organism, such as an animal or a human.

The nature of the sample in which detection of an analyte is envisaged according to the present invention is not critical, and may be any preparation suspected to comprise nucleic acids. The sample may be a sample comprising biological material such as, but not limited to, body tissue or fluid such as blood, saliva, semen, stool or urine, as well as compositions derived or extracted from such biological material. The sample may comprise components of biological material such as cells, e.g. tissue cells, red blood cells, white blood cells, platelets, dead cells. The material can be obtained from an organism by way of swabs, including but not limited to buccal swabs, throat swabs, vaginal swabs, urethral swabs, cervical swabs, throat swabs, rectal swabs, lesion swabs, abcess swabs, nasopharyngeal swabs, and the like. The sample can comprise a body fluid such as lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, plasma, serum, pulmonary lavage, lung aspirates, or part or component thereof. The sample may be obtained from biopsy material of any tissue in the body. In addition, tissue culture cells, including explanted material, primary cells, secondary cell lines, and the like, as well as lysates, extracts, supernatants or materials obtained from any cells, tissues or organs, are also considered to fall within the meaning of the term sample as used herein. Samples comprising microorganisms or viruses are also envisaged in the context of nucleic acid detection using the methods of the invention. Materials obtained from forensic settings are also within the intended meaning of the term “sample”. Samples may also comprise foodstuffs and beverages, environmental samples such as water, soil, sand, etc. These lists are not intended to be exhaustive.

In a particular embodiment of the invention, the sample is pre-treated to facilitate the detection of the analyte with the detection method. Extraction of nucleic acids such as DNA or RNA is a typical pre-treatment of the sample envisaged in the context of the present invention. Methods and kits suitable for extracting nucleic acids are available in the art and include methods and kits based on phenol-chloroform extraction, salting out DNA extraction, and guanidinium thiocyanate extraction.

Exemplary nucleic acid isolation techniques include (1) organic extraction followed by ethanol precipitation, e.g. using a phenol/chloroform organic reagent (e.g. Ausbel et al., eds., (1995, including supplements through June 2003) Current Protocols in Molecular Biology, John Wiley & Sons, New York), preferably using an automated DNA extractor, e.g. the Model 341 DNA Extractor available from Applied Biosystems (Foster City, Calif.), (2) stationary phase adsorption methods (e.g. Boom et al., U.S. Pat. No. 5,234,809; Walsh et al., BioTechniques 10(4): 506-513 (1991), and (3) salt-induced DNA precipitation methods (e.g. Miller et al., (1988) Nucl. Acids Res., 16(3):9-10), such precipitation methods being typically referred to as “salting-out” methods. Commercially available kits can be used to expedite such methods, for example, Genomic DNA Purification Kit and the Total RNA Isolation System (both available from Promega, Madison, Wis.). Further, such methods have been automated or semi-automated using, for example, the ABI PRISM™ 6700 Automated Nucleic Acid Workstation (Applied Biosystems, Foster City, Calif.) or the ABI PRISM™ 6100 Nucleic Acid PrepStation and associated protocols, e.g. NucPrep™ Chemistry: Isolation of Genomic DNA from Animal and Plant Tissue, Applied Biosystems Protocol 4333959 Rev. A (2002), Isolation of Total RNA from Cultured Cells, Applied Biosystems Protocol 4330254 Rev. A (2002), and ABI PRISM™ Cell Lysis Control Kit, Applied Biosystems Protocol 4316607 Rev. C (2001).

The above pre-treatment methods can further comprise a fragmentation step, e.g. by enzyme digestion, shearing or sonication, and/or an enzymatic amplification step, e.g. by PCR including RT-PCR. Most particularly, where sensitive detection of a nucleic acid is envisaged, a PCR amplification of the target DNA can be performed prior to the detection of the analyte. Thus, according to this embodiment, the sample on which detection is performed comprises an amplified PCR product.

The methods of the present invention are designed for the detection of single-stranded (ss) nucleic acids. Therefore double-stranded (ds) nucleic acids, in order to be detected by the method of the present invention, need to be denatured so that the two strands separate into single nucleotide strands. DNA denaturation methods include incubation at a high temperature (above the DNA's melting point, generally a temperature of 95° C. is used resulting in the simultaneous inactivation of most enzymatic activity present in the sample), immediately followed by chilling on ice to prevent renaturation of the strands. DNA can also be denatured by low salt concentrations or high pH. Several methods have been described for the generation of ssDNA from dsDNA of PCR products. These methods include asymmetric PCR, PCR with chemically modified primers that causes the synthesis of strands of unequal length, and preferential exonuclease digestion of one appropriately modified strand, followed by a purification step to isolate the ssDNA (Pragratis N.C., 1996, Nucl. Acids Res., 24:3645-3646).

The present invention provides a displacement assay that involves detection and/or quantification using a label. While the methods of the invention make use of the specific properties of the combined signal of a SE(R)RS label and a SE(R)RS surface, it is envisaged that the detection and/or quantification of the analyte in the sample can be performed using either SE(R)RS-based detection or detection of fluorescence of the labels, or using both SE(R)RS-based and fluorescence-based detection. This feature of the methods of the present invention is based on the fact that a) most SE(R)RS labels are also fluorescent, and visa versa, and b) that there is a comparable but inverse effect of the proximity of a SE(R)RS surface on the SE(R)RS and fluorescence signal detected from a label.

SE(R)RS labels generally benefit from signal intensity enhancement in the proximity of a metal surface. When the labels are adsorbed onto or near the SE(R)RS surface with a distance at which their Raman scattering is enhanced, the emitted fluorescence of the label is quenched by the metal surface. When the labels are positioned further away from the surface, fluorescence, but not Raman scattering, is enhanced.

When the distance is further increased, the enhancement of the fluorescence also dies out. Thus, when the label is in close proximity to the SE(R)RS surface, the SE(R)RS signal is enhanced, and the fluorescent signal is quenched. On the other hand, when the label is separated from the proximity of the SE(R)RS surface, the SE(R)RS signal dies out, while the fluorescence signal can still be detected.

Where the detection of the analyte in the methods of the invention is based on SE(R)RS, the detection of the SE(R)RS signal and/or its intensity is inversely proportionate to the amount of analyte in the sample. Indeed, as the signal is provided by the complex of the hybridized displaceable surrogate target probe and capture probe the so-called “surrogate hybrid”), an increasing concentration of analyte in the sample will result in increased displacement of the displaceable surrogate target probe and a decrease in SE(R)RS signal.

Furthermore, SE(R)RS detection of the analyte in the methods of the invention requires a label which is a SE(R)RS-active material, i.e. capable of generating a SERS or SERRS spectrum when appropriately illuminated, also referred to herein as a SE(R)RS label or dye. Non-limiting examples of SE(R)RS labels include fluorescein dyes, such as 5- (and 6-) carboxy-4′,5′-dichloro-2′,7′-dimethoxy fluorescein, 5-carboxy-2′,4′,5′,7′-tetrachlorofluorescein and 5-carboxyfluorescein, rhodamine dyes such as 5- (and 6-) carboxy rhodamine, 6-carboxytetramethyl rhodamine and 6-carboxyrhodamine X, phthalocyanines such as methyl, nitrosyl, sulphonyl and amino phthalocyanines, azo dyes, azomethines, cyanines and xanthines such as the methyl, nitro, sulphano and amino derivatives, and succinylfluoresceins. Each of these may be substituted in any conventional manner, giving rise to a large number of useful labels. It is noted that the choice of the label can be influenced by factors such as the resonance frequency of the label, the resonance frequency of other molecules present in the sample, etc. SE(R)RS labels of use for detecting biomolecules are described in the art such as in U.S. Pat. Nos. 5,306,403, 6,002,471, and 6,174,677.

Where the detection of the label is based on fluorescence, the detection of the signal and/or its intensity is directly proportionate to the amount of analyte in the sample. Indeed, as the fluorescence signal of the label bound to the displaceable surrogate target probe is only detected when it is not bound to (or displaced from) the capture probe, an increasing concentration of analyte in the sample will result in increased displacement of the displaceable surrogate target probe and an increase in fluorescence signal.

Where the detection of the label is based on fluorescence, fluorescent labels are used. Fluorescent labels include but are not limited to fluorescein isothiocyanates (FITC), carboxyfluoresceins such as tetramethylrhodamine (TMR), carboxy tetramethyl-rhodamine (TAMRA), carboxy-X-rhodamine (ROX), sulforhodamine 101 (Texas red™), Fluorescent Red and Fluorescent Orange, phycoerythrin, phycocyanin, and Crypto-Fluor™ dyes, etc.

According to a particular embodiment, the methods provide for detection of both fluorescence and SE(R)RS detection. Methods which comprise detection of both the fluorescence and the SE(R)RS signal of the label of the surrogate target probe provide an additional control on accuracy, as they allow both a direct and indirect measurement of the presence and/or quantitity of the analyte in the sample. In these methods a label is used which is suitable for both detection methods. Most particularly, the methods of the invention are performed using a label selected from the group consisting of HEX (2,5,1′,3′,7′,9′,-Hexachloro-6-carboxyfluorescein), fluorescein, and TET (6-carboxy-2′,4,7,7′-tetrachlorofluorescein).

According to the present invention, the label is attached to a surrogate target probe, which can be either a displaceable or a non-displaceable surrogate target probe. Attachment of a label to an oligonucleotide can be performed by methods as described in the prior art (e.g. U.S. Pat. No. 6,127,120). Other methods for preparing labeled oligonucleotides by incorporating labels into oligonucleotides are described in e.g. U.S. Pat. Nos. 4,962,037, 5,405,747, 6,136,543, and 6,210,896. In a particular embodiment of the invention, a SE(R)RS label is used, which is either attached directly to the nucleotide probe or via a linker compound. SE(R)RS labels that contain reactive groups designed to covalently react with other molecules, such as nucleotides or nucleic acids, are commercially available (e.g. Molecular Probes, Eugene, Oreg.). SE(R)RS labels that are covalently attached to nucleotide precursors may be purchased from standard commercial sources (e.g. Roche Molecular Biochemicals, Indianapolis, Ind.; Promega Corp., Madison, Wis.; Ambion, Inc., Austin, Tex.; Amersham Pharmacia Biotech, Piscataway, N.J.).

According to the present invention, detection is performed by providing a displaceable surrogate target probe which is labeled and comprises a sequence similar to the target sequence within the analyte of interest such that competition with the analyte for binding to the capture probe is enabled. The ability of the analyte to compete with and displace the displaceable surrogate target probe is ensured by providing a surrogate target probe which binds to the capture probe with a strength that is lower than if the analyte were bound to the capture probe. According to a particular embodiment, the melting temperature (Tₘ), defined as the temperature at which 50% of the hybrids are denatured into single strands, of the surrogate hybrid is lower than that of the target hybrid. Typically, this is ensured by introducing one or more modifications in the nucleotide sequence of the displaceable surrogate target probe which affect its binding to the capture probe. Suitable modifications include the introduction of one or more single nucleotide polymorphism (SNP), insertions (e.g. of hairpin structures), etc. A number of different ways to calculate the melting temperature of an oligonucleotide are described in the art and services calculating Tₘ based on algorithms are provided by different sources on the internet (e.g. by Stratagene: www.stratagene.com/QPCR/tnCalc.asp). These methods make it possible to evaluate the effect of the incorporation of one or more mismatches in the sequence of the displaceable target probe on the Tₘ. While each of these methods may give slightly different results, and optimum Tₘ value must be determined empirically, most of these methods are useful to provide an indication of the suitability of the displaceable target probe in the context of the present invention.

The difference in melting temperature between the oligonucleotide sequence of a displaceable surrogate target probe and a target sequence will correspond to a difference in optimal annealing temperature. If the annealing temperature is set too high a nucleotide sequence (e.g. the probes, the analyte) will not anneal efficiently, and if the annealing temperature is set too low a nucleotide sequence (e.g. the probes, the analyte) may anneal nonspecifically. When performing the methods of the present invention, in order to ensure the displacement of the displaceable surrogate target probe by the analyte, the contacting of the analyte with the capture probe and the displaceable surrogate target probe (which have optionally been allowed to form a surrogate hybrid) will be performed at an annealing temperature which is the same or preferably higher than the melting temperature of the displaceable surrogate target probe. Thus, according to one embodiment, the contacting step can be performed at the melting temperature of the displaceable surrogate target probe, more particularly at an optimal annealing temperature which is between 0-10° C. higher than the melting temperature of the displaceable surrogate target probe, most particularly at an optimal annealing temperature which is between 0-5° C. higher than the melting temperature of the displaceable surrogate target probe.

It can be envisaged that the contacting step of the displaceable surrogate target probe and the capture probe comprises an annealing step at an annealing temperature which is optimally 2-6° C. lower than the melting temperature of the surrogate hybrid.

It can also be envisaged that the contacting step of the analyte with the capture probe and the displaceable surrogate target probe (which have optionally been allowed to form a surrogate hybrid) comprises a denaturation step (typically heating to about 90-95° C.) and thereafter an annealing step which is performed at the optimal annealing temperature of the target sequence (which will be higher than the optimal annealing temperature of the displaceable surrogate target probe, see above). A denaturation step can be of interest when the analyte is double stranded and denaturation of the double stranded DNA is required to allow detection. A denaturation step can further be of interest when not only qualitative but also quantitative information of the analyte(s) in the sample is required.

In a particular embodiment, the surrogate hybrid is designed such that its Tₘ is about 15 to 20° C. lower than the Tₘ of the target hybrid. This may e.g. be feasible by including several mismatches in the oligonucleotide moiety of the surrogate target probe. As a result, the surrogate hybrid will be completely melted at a temperature where the target hybrid remains conserved i.e. the capture probe remains annealed to the target sequence. This approach enables the use of one single melting step at moderate temperature.

In the methods of the present invention, the detection of the signal of the label present on the displaceable surrogate target probe can be monitored “real time” during melting and/or annealing.

The contacting of the different reagents used in the methods of the present invention can be varied, depending on the nature of the detection signal (and information obtainable therefrom) desired. According to a particular embodiment of the invention, the capture probe and the displaceable surrogate target probe are contacted first, and allowed to form a surrogate hybrid. Upon addition of the SE(R)RS surface to this hybrid, the signal of this surrogate hybrid can be detected and serves as a baseline detection signal. The sample is then contacted with the surrogate hybrid and the displaceable surrogate target probe is displaced from the capture probe by the analyte present in the sample. Detection of the signal of the remaining surrogate hybrid is indicative of the amount of analyte in the sample. Alternatively, the sample, the surrogate target probe, and the capture probe can be contacted simultaneously. According to yet a further embodiment, the sample is contacted with the surrogate hybrid prior to the addition of the SE(R)RS surface. In the latter two embodiments, there is no detection of the baseline value, and only an indication of the label signal in the presence of analyte is obtained. However, the baseline value can be determined if a separate measurement is performed, e.g. in a control sample, e.g. before measuring a large number of similar samples. Alternatively, a baseline value can be determined if an internal standard (as described below) comprising a different SE(R)RS label is introduced in the sample. Furthermore, if only qualitative information is required, detection of fluorescence (due to the displaced surrogate target probe) can be used to confirm the presence of the target sequence(s) in the sample. According to a particular embodiment of the invention, the capture probe is covalently bound to the SE(R)RS surface , removing the need for an additional step comprising the adsorption to the SE(R)RS surface. As indicated above, the detection of the SE(R)RS signal can in most cases be complemented or substituted by detection of a fluorescent signal.

In a further aspect of the invention methods are provided wherein the detection of the analyte is based on competitive SE(R)RS as described above, whereby a(n) (internal) standard hybrid is included for calibrating the assay with respect to aggregate and optical variations. According to this aspect, the method further encompasses the step of adding a capture probe and a labeled probe complementary thereto which are the standard probe and the standard capture probe, able to form a “standard hybrid”. According to a particular embodiment, the standard hybrid is an internal standard, i.e. added to the sample. In order to be able to discriminate the signal of the standard hybrid from that of the surrogate hybrid in the sample, a different SE(R)RS (and/or fluorescent) label is used. According to another particular embodiment, the standard hybrid is added to a portion of the sample. According to yet another particular embodiment, the standard hybrid is added to the control sample. The latter two embodiments may be particularly useful when measuring a large number of similar samples.

According to one embodiment, the sequence of the standard capture probe is the same as that of the capture probe used in detection, and the standard probe is a non-displaceable surrogate target probe. As the Tₘ of the non-displaceable surrogate target probe is higher than that of the displaceable surrogate target, the non-displaceable surrogate target probe is not displaced from the capture probe by the analyte at the Tₘ of the surrogate target probe. Alternatively, the nucleotide sequence of the standard capture probe is not target-specific but an (arbitrary) sequence which specifically binds a corresponding standard probe. This avoids any interaction of the standard hybrid with the detection of the analyte even when incorporating a higher temperature step such that the addition of the standard hybrid to the sample can be performed at any time prior to detection (or addition of SE(R)RS surface, where required) and either as a hybrid or as its individual components. The provision of a standard probe and standard capture probe combination which are different from the analyte moreover allows the use of the same standard hybrid in different detections.

According to the present invention, a displacement assay is described which makes use of the specific features of surface-enhanced spectroscopy (and optionally its inherent effect on fluorescence). Detection by surface-enhanced spectroscopies such as SE(R)RS is based on the strong enhancement of Raman scattering observed for analytes adsorbed onto or in close (≦±30 Å) proximity to a roughened metal surface. According to the present invention, a SE(R)RS label is detected by addition of a suitable SE(R)RS surface. Typically, the surface is a noble (Au, Ag, Cu) or alkali (Li, Na, K) metal surface. The metal surface may for instance be an etched or otherwise roughened metallic surface, a metal sol or according to a particular embodiment, an aggregation of metal colloid particles as the latter can ensure an enhancement of greater than 10⁸-10¹⁴ of the Raman scattering. The metal nanoparticles making up the SE(R)RS surface in the detection methods of the present invention can also be arranged in metal nanoparticle island films, metal-coated nanoparticle-based substrates, polymer films with embedded metal nanoparticles, and the like. The metal surface may be a naked metal or may comprise a metal oxide layer on a metal surface. It may include an organic coating such as of citrate or of a suitable polymer, such as polylysine or polyphenol, to increase its sorptive capacity.

According to a particular embodiment of the invention, the metal colloid particles making up the SE(R)RS surface are nanoparticles or colloidal nanoparticles aggregated in a controlled manner such as described in US 2005/0130163 A1. Alternative methods of preparing nanoparticles are known (e.g. U.S. Pat. Nos. 6,054,495, 6,127,120, and 6,149,868). Nanoparticles may also be obtained from commercial sources (e.g. Nanoprobes Inc., Yaphank, N.Y.; Polysciences, Inc., Warrington, Pa.). The metal particles can be of any size as long as they give rise to a SE(R)RS effect. Typically they have a diameter of about 4-50 nm, most particularly between 25-40 nm, depending on the type of metal.

According to another particular embodiment, the SE(R)RS surface is a colloidal suspension of silver or gold nanoparticles, or aggregated colloids thereof. An optimal size and shape of the aggregated colloid particles which can be critical for reproducibility and calibration of the detection, can be ensured by using a polyamine such as poly-L-lysine or spermine. Since the size of the aggregated colloids may change with time, they are ideally formed in situ in the detection sample and the SE(R)RS spectrum should be obtained shortly afterwards (preferably within about 15 minutes of aggregation).

According to a further particular embodiment, the SE(R)RS surface consists of gold nanoparticles that are coated with silver by addition of silver hydroquinone after covalently linking the capture probe to the gold nanoparticles. Most particularly, according to this embodiment, the SE(R)RS surface consists of silver or gold nanoparticles coated with a thin layer (1 to a few nanometers) of gold or silver, respectively.

According to yet another particular embodiment, the SE(R)RS surface consists of stable clusters of silver or gold nanoparticles and the clusters are established by cross-linking with bi- or multifunctional macromolecules (telemers) which can bind chemically to said clusters.

In the detection and/or quantification methods of the present invention making use of either or both SE(R)RS and fluorescence detection, the label of the surrogate target probe is brought into close proximity with the SE(R)RS surface by binding the surrogate target probe to the capture probe, the latter being linked to the SE(R)RS surface. Binding of the capture probe to the SE(R)RS surface can be through a covalent or non-covalent binding. Various options and modes for binding oligonucleotide probes to SE(R)RS surfaces are known in the art (U.S. Pat. Nos. 6,127,12 and 6,972,173) and can be applied for binding the capture probe or the surrogate hybrid to the SE(R)RS surface.

According to one embodiment, the attachment of the capture probe to the SE(R)RS surface is through a covalent binding. The capture probe is fitted with a surface-seeking group so as to promote or facilitate chemi-sorption of the probe onto the SE(R)RS surface. Surface-seeking groups are soluble in solution but interact preferentially with the SE(R)RS surface. This can be ensured by incorporating one or more functional groups into the capture probe's oligonucleotide moiety. Suitable functional groups include the Lewis bases such as thiols and amines which can easily be added to nucleic acids. Other examples of surface-seeking groups include complexing groups such as nitrogen, oxygen, sulphur and phosphorous donors, chelating groups, bridging ligands and polymer forming ligands. For gold surfaces phosphorus and sulphur containing groups may be particularly preferred.

According to a particular embodiment the capture probe's surface-seeking group is a benzotriazole enabling the capture probe to be covalently linked to the SE(R)RS surface.

According to another particular embodiment the capture probe's surface-seeking group consists of at least one thiol group enabling the capture probe to be covalently linked to the SE(R)RS surface.

A preferred functional group is one which can provide additional cationic sites (under the conditions used), e.g. one comprising an amino, preferably a primary amino group or groups. According to a preferred embodiment the capture probe's surface-seeking group comprises positively charged propargylamine moieties. According to another embodiment, adsorption of the capture probe to the metal SE(R)RS surface is ensured by addition of a monomeric or polymeric polyamine, more particularly a short-chain aliphatic polyamine such as spermine. Thus, according to one embodiment, the methods of the invention will comprise, prior to detection, addition of a polyamine to the sample. The polyamine should be introduced at a time which allows its interaction with the surrogate hybrid, before the SE(R)RS spectrum is obtained. The polyamine is preferably a short-chain aliphatic polyamine such as spermine, spermidine, 1,4-diaminopiperazine, diethylenetriamine, N-(2-aminoethyl)-1,3-propanediamine, triethylenetetramine and tetraethylenepentamine. Spermine with its four NH₂ groups per repeat unit is particularly suitable for use in the present invention. The polyamine is preferably introduced in the form of an acid salt such as its hydrochloride. It is of most use when the SE(R)RS surface is colloidal (see above). The amount of polyamine added is preferably of the order of 100 to 1000 times more than would be needed to obtain a monolayer coverage of the surface with the polyamine. Excess polyamine forms a coating on the surface thereby ensuring optimal colloidal aggregation and adsorption of capture probe and/or surrogate hybrid.

As indicated above, the methods of the present invention are of particular interest in detection and/or quantification methods based on surface-enhanced (resonance) Raman spectroscopy or SE(R)RS. Though reference is generally made to SE(R)RS herein, it will be understood that detection methods based on other types of surface-enhanced spectroscopies are also envisaged, for example, but not limited to, surface-enhanced fluorescence, normal (Stokes or anti-Stokes) Raman scattering, resonance Raman scattering, coherent (Stokes or anti-Stokes) Raman spectroscopy (CSRS or CARS), Surface-enhanced (resonance) CARS, stimulated Raman scattering, inverse Raman spectroscopy, stimulated gain Raman spectroscopy, hyper-Raman scattering, surface-enhanced hyper-Raman scattering, molecular optical laser examiner (MOLE) or Raman microprobe or Raman microscopy or confocal Raman microspectrometry, three-dimensional or scanning Raman, Raman saturation spectroscopy, time-resolved resonance Raman, Raman decoupling spectroscopy or UV-Raman microscopy.

In a particular embodiment of the invention, the detection method of the invention involves SERRS, since operating at the resonant frequency of the label gives increased sensitivity. In this case, the light source used to generate the Raman spectrum is a coherent light source, e.g. a laser, tuned substantially to the maximum absorption frequency of the label being used. This frequency may shift slightly on association of the label with the SE(R)RS surface and the oligonucleotide moiety of the surrogate target probe, but the skilled person will be well able to tune the light source to accommodate this. The light source may be tuned to a frequency near to the label's absorption maximum, or to a frequency at or near that of a secondary peak in the label's absorption spectrum. SERRS may alternatively involve operating at the resonant frequency of the plasmons on the active surface or (aggregated) colloids.

In the methods of the invention based on SE(R)RS detection, typically one peak, corresponding e.g. to the label's absorption maximum, is selected and excitation is performed only at the wavelength of that peak. Alternatively, where e.g. different analytes are being detected at the same time using different labels, it may be necessary to detect the entire “fingerprint” spectrum in order to identify each label. In general multivariate analysis methods (such as partial least squares regression, principal components regression, etc.) may be used to perform qualitative and/or quantitative identification of each of the labels present, using either the entire fingerprint spectrum, a spectral range with more than one Raman band, or using one unique Raman band.

In a particular embodiment of the invention, the detection method of the invention involves the simultaneous detection by both surface-enhanced (resonance) Raman and fluorescence spectroscopy (see above).

Typically, the detection step in a SE(R)RS based detection method will be carried out using incident light from a laser, having a frequency in the visible spectrum. The exact frequency chosen will depend on the label, surface and analyte. Frequencies in the green or red area of the visible spectrum tend, on the whole, to give rise to better surface enhancement effects for noble metal surfaces such as silver and gold. However, it is possible to envisage situations in which other frequencies, for instance in the ultraviolet or the near infrared ranges, might be used. The selection and, if necessary, tuning of an appropriate light source, with an appropriate frequency and power, will be well within the capabilities of one of ordinary skill in the art, particularly on referring to the available SE(R)RS literature.

Excitation sources for use in SE(R)RS-based detection methods include, but are not limited to, nitrogen lasers, helium-cadmium lasers, argon ion lasers, krypton ion lasers, etc. Multiple lasers can provide a wide choice of excitation lines which is critical for resonance Raman spectroscopy. According to a specific embodiment, an argon ion laser is used in a LabRam integrated instrument (Jobin Yvon) at an excitation wavelength of 514.5 nm.

The excitation beam may be spectrally purified with a bandpass filter and may be focused on a substrate using a 6 times objective lens. The objective lens may be used to both excite the sample and to collect the Raman signal, by using a holographic beam splitter to produce a right-angle geometry for the excitation beam and the emitted Raman signal. The intensity of the Raman signals needs to be measured against an intense background from the excitation beam. The background is primarily Rayleigh scattered light and specular reflection, which can be selectively removed with high efficiency optical filters. For example, a holographic notch filter may be used to reduce Rayleigh scattered radiation.

The surface-enhanced Raman emission signal may be detected by a Raman detector. A variety of detection units of potential use in Raman spectroscopy are known in the art and any known Raman detection unit may be used. An example of a Raman detection unit is disclosed e.g. in U.S. Pat. No. 6,002,471. Other types of detectors may be used, such as a charge coupled device (CCD), with a red-enhanced intensified charge-coupled device (RE-ICCD), a silicon photodiode, or photomultiplier tubes arranged either singly or in series for cascade amplification of the signal. Photon counting electronics can be used for sensitive detection. The choice of detector will largely depend on the sensitivity of detection required to carry out a particular assay. Several devices are suitable for collecting SE(R)RS signals, including wavelength selective mirrors, holographic optical elements for scattered light detection and fibre-optic waveguides. Various options are discussed, e.g. in WO 97/05280.

An apparatus for obtaining and/or analysing a SE(R)RS spectrum may include some form of data processor such as a computer. Once the SE(R)RS signal has been captured by an appropriate detector, its frequency and intensity data will typically be passed to a computer for analysis. Either the fingerprint Raman spectrum will be compared to reference spectra for identification of the detected Raman active compound or the signal intensity at the measured frequencies will be used to calculate the amount of Raman active compound detected.

A particular embodiment of the present invention provides SE(R)RS displacement assays for detecting two PCR products, gene A and gene B within a sample. The methods are essentially as follows. The capture probes for gene A and gene B, and their corresponding surrogate target probes carrying the labels HEX and TET, respectively, are contacted and allowed to hybridize. The capture probes contain surface seeking groups which promote adsorption onto the silver surface. Hybridization of surrogate target probes and capture probes yields the surrogate hybrids for each of the target sequences of the genes A and B. The surrogate hybrids are subsequently adsorbed onto silver nanoparticle colloids. A first SE(R)RS measurement is performed. A sample containing the amplified genes A and B is added. After denaturation e.g. at 95° C. so as to dissociate the surrogate targets and the double-stranded PCR products, the temperature is lowered to an annealing temperature which is optimal for annealing the analyte DNA's (i.e. denatured genes A and B) to their corresponding capture probes, and sub-optimal for annealing the surrogate target probes to these probes. In this way, competition between the analyte DNA's and the surrogate target probes will take place and will result in the surrogate target probes being effectively displaced from the surface-adsorbed surrogate hybrid. As discussed above, displacement conditions will be optimal at an annealing temperature that is higher than the Tₘ of the surrogate hybrid. A second SE(R)RS measurement will yield a lower SE(R)RS signal than prior to addition of the analytes because the SE(R)RS label, attached to the displaced surrogate target probe, is no longer in close enough proximity to the SE(R)RS surface. Optionally, fluorescence is measured along with or instead of the SE(R)RS. The fluorescence of the label in the surrogate hybrid is close to zero due to quenching. If the analyte is present in the sample, the signal will increase accordingly, as displacement of the surrogate target probe removes the source of quenching. The amount surrogate target probe displaced for each of the genes is indicative of the amount of genes A and B present in the sample.

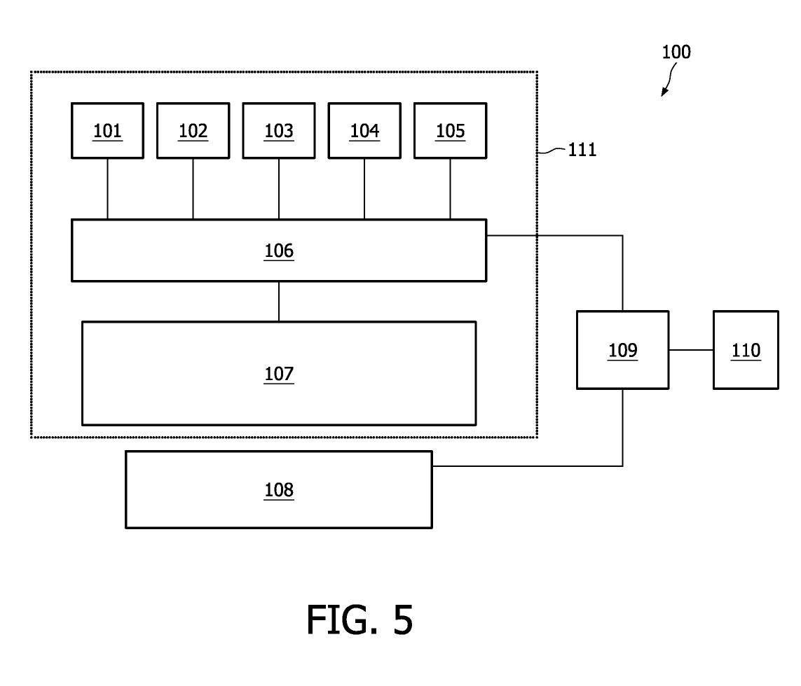

FIG. 5 is a schematic representation of the system 100 according to an embodiment of the present invention. The system 100 is suitable for detecting and optionally quantifying the presence of at least one analyte in a sample using the displacement assay based on SE(R)RS and fluorescence. It comprises a source 101 for providing a sample, at least one source 102 for providing a surrogate target probe, at least one source 103 for providing a capture probe, and optionally, at least one source 104 for providing the reagents for the internal standard and at least one source 105 of additives serving in the detection. The device further comprises a means 107 wherein the surrogate target probe(s), the capture probe(s) and the analyte(s) are contacted and presented for detection. Contacting means 107 also includes a heating facility for ensuring appropriate temperatures in the contacting means.

The device further comprises means 106 for

a) providing surrogate target probe(s) and capture probe(s) from sources 102 and 103, respectively, to means 107 for contacting the surrogate target probe(s) with the capture probe(s) to generate a surrogate hybrid(s),
b) providing additives from source 105 to means 107 for contacting the surrogate hybrid(s) with a SE(R)RS surface to enable detection of the SE(R)RS label attached to the surrogate target probe(s),
c) providing sample comprising analyte(s) from source 101 to means 107 for contacting the sample with the surrogate hybrid(s) to enable displacement of the surrogate target probe(s), and optionally,
d) providing at least one internal standard reagent from at least one source 104 to means 103 for calibrating the assay with respect to variations of aggregate formation and optical variations.

The means 106 may include gravimetric feeds of the sample and/or analyte and may also include an arrangement of pipes/conduits and valves, e.g. selectable and controllable valves, to allow the provision of the fluids from sources 101, 102, 103, 104, and 105 to the contacting means 107. Alternatively, the fluids may be pumped from the sources 101-105 to the contacting means 107.

The above arrangement of components may be located on a cartridge 111, e.g. a disposable cartridge 111 for use in molecular diagnostics.

Control and analysis circuitry 109, which may be at least partly in the cartridge 111 or may optionally be external to the cartridge 111 and may be provided optionally to control the operation of the means 106. The control and analysis circuitry 109 may be connected to the means 106 by suitable contacts on the surface of the cartridge, e.g. terminals.

Further, means 108 for detecting the labels are provided. Means 108 may be integral with the cartridge 111 or may be external to the cartridge and windows may be provided in the cartridge 111 so that the detection means 108 may detect the sample, etc. The means 108 may be under the control of the control and analysis circuitry 109. The detection means 108 may be a detector able to use SE(R)RS and fluorescence detection methods as mentioned above. The means 108 may include excitation source, a filter and a detector. Typically the means for detecting comprises a spectrophotometric device. Signals representative of the detections may be supplied to the control and analysis circuitry 109 which can be adapted to carry out any of the analysis algorithms of the present invention described above. The results may be displayed on any suitable display means 110 such as a visual display unit, plotter, printer, etc. The control and analysis circuitry 109 may have a connection to a local area or wide area network for transmission of the results to a remote location. Control and analysis circuitry 109 may be implemented in any suitable manner, e.g. dedicated hardware or a suitably programmed computer, microcontroller or embedded processor such as a microprocessor, programmable gate array such as a PAL, PLA or FPGA, or similar.

In accordance with a specific embodiment of the present invention the sample in source 101 may be a solution containing DNA obtained from a PCR reaction. This embodiment is particularly useful for use in a molecular diagnostic disposable cartridge which can include a cell lysis station and a PCR reaction station, in particular a multiplexed PCR reaction station. The output of the PCR reaction station then forms the source 101. The capture probe in source 103 for detection of an amplified oligonucleotide is an analyte-specific oligonucleotide probe. The surrogate target probe in source 102 is an oligonucleotide probe to which a SE(R)RS label is attached which is suitable for both SE(R)RS and fluorescence detection. According to a particular embodiment, the nucleotide sequence of the surrogate target probe contains a SNP such that the binding strength of the surrogate target probe to the capture probe is sub-optimal. Source 104 may contain a predetermined amount of internal standard being a (non-displaceable) standard hybrid. A first source 105 may contain a suitable metal surface such as colloid particles or beads coated with metal surface, especially aggregatable colloid particles or beads coated with metal surface. A second source 105 may contain an aggregating agent such as spermine.

In this specific embodiment, appropriately providing and contacting of reagents from the sources 101 to 105 results in contacting means 107 containing surface-adsorbed surrogate hybrid and surface-adsorbed standard hybrid. A first measurement is performed at this point as a reference. In a next step, sample is added. Upon displacement of the surrogate target probe from the capture probe, a decrease in SE(R)RS signal and/or an increase in fluorescence is observed.

Other arrangements of the systems and methods embodying the invention will be obvious for those skilled in the art.

Yet a further aspect of the invention provides the use of the systems, disposable cartridges, combinations, and methods of the present invention in the detection and/or quantification of an analyte in a sample in the context of applications such as but not limited to medical diagnostics such as molecular diagnostics of infectious diseases, pathology, toxicology, epidemiology, biological warfare, environmental sampling, forensics, etc.

It is to be understood that although preferred embodiments, specific constructions and configurations, as well as materials, have been discussed herein for devices according to the present invention, various changes or modifications in form and detail may be made without departing from the scope and spirit of this invention.

### EXAMPLE 1

### Detection and Quantification of Specific Genes in a Sample using Competitive SERRS with Internal Calibration

Highly pathogenic avian influenza caused by certain subtypes of influenza A virus in animal populations, particularly chickens, poses a continuing global human public health risk. Direct human infection by the avian influenza A subtype H5N1 virus has been responsible for considerable human mortality recently, stressing the need for rapid and accurate diagnosis. Type A influenza viruses are subtyped on the basis of antigenic differences in the external glycoproteins, the hemagglutinins (HA) and the neuraminidases (NA). The present invention offers a novel, rapid and accurate approach to viral subtyping by using RT-PCR and SERRS of viral nucleic acids.

Viral RNA is extracted from clinical samples and cDNA complementary for viral RNA is generated using viral reverse transcriptase and random primers according to Wright et al. (1995), J. Clin. Microbiol., 33:1180-1184. Multiplex PCR is carried out with two sets of primers specific for the HA and NA genes of influenza virus subtype H5N1, as described in “Recommended laboratory tests to identify avian influenza A virus in specimens from humans”, WHO Geneva, June 2005), designed to yield PCR products of 219 and 616 bp, respectively. Amplified products are subsequently detected by competitive SERRS.

Thereto, two analyte-specific probes, so-called capture probes, are designed to be complementary to a region within the HA and NA genes, respectively, and having the surface-seeking group consisting of positively charged propargylamine moieties for attachement to a silver nanoparticle. Two additional synthetic oligonucleotides, so-called surrogate targets, labeled with the SERRS dyes HEX and TET, respectively, are designed to be complementary to a portion of the HA- and NA-capture probes, respectively, except for one mismatch (SNP).

Upon contacting the surrogate target probes with the corresponding capture probes, surrogate hybrids are formed. However, due to the presence of the mismatch in the sequence of the surrogate target probes, the surrogate target probes and their corresponding HA- and NA-capture probes are loosely annealed.

A predetermined amount of internal standard which is a non-displaceable surrogate hybrid carrying a label which is different from the label used for detection, is added to the above mixture for calibration and to correct for possible variations in aggregate formation and excitation intensity.

A first detection step determines reference points for the SERRS measurement of HEX and TET and a reference point for the internal calibration. Thereto, the above prepared solution is mixed with 10 μl spermine tetrachloride (100 mM in water, freshly prepared) followed by 250 μl water and 250 μl citrate-reduced silver nanoparticles (prepared as described by Munro et al. (1995), Langmuir, 11:3712-3720). Adsorption of the surrogate hybrids to the silver colloids will bring the HEX and TET dyes in close enough proximity to the metal surface for enabling SERRS detection while fluorescence is quenched. Likewise, adsorption of the internal standard to the silver colloids will enable SERRS detection of the internal standard's dye. Immediately after mixing the SERRS spectrum is taken from the prepared solution using a LabRam system (Jobin Yvon) with an Argon laser providing excitation at 514.5 nm.

For detection of amplified H5N1 viral HA and NA genes the output of the PCR reaction is added. After denaturation at 95° C., incubation at an appropriate annealing temperature results in the association of the analyte DNA's with the HA- and NA-capture probes, the latter being attached to the aggregated silver colloids. Since the sequence of the analytes is perfectly complementary to the HA- and NA-specific capture probes, hybridization of the analyte DNA's to the HA- and NA-capture probes results in target hybrids which cannot be displaced at the melting temperature of the surrogate hybrids. Provided the Tₘ of the standard hybrid is higher than that of the surrogate hybrids, the standard hybrid is unaffected by the above incubation step.

Immediately after binding of the analyte DNA's with the capture probes and within a time scope of 15 min after the first SERRS measurement, the second SERRS spectrum is taken. The intensity of the SERRS signal of the HEX and TET dyes is now reduced due to the displacement of the surrogate target probes, to which the dyes are attached, from the metal surface. The amount of HA or NA genes in the PCR output can now be calculated as it corresponds to the amount of surrogate target probes displaced. Thereto, the first and the second SERRS measurements need to be compared.

For calibration, the first and second SERRS spectrum of the internal standard is compared and intensity variations are corrected for.

This example demonstrates that an accurate detection of amplified viral HA and NA genes using competitive SERRS can be achieved without the need for labeling the amplified genes.

1. A method for detecting the presence and/or quantity of at least one analyte in a sample comprising the steps of:
(a) contacting said sample with:
(i) at least one target-specific capture probe comprising:
an oligonucleotide capable of specifically binding a target sequence within said analyte, and
(ii) at least one displaceable surrogate target probe comprising:
an oligonucleotide capable of specifically binding said capture probe,
a label which has either or both surface-enhanced (resonance) Raman scattering (SE(R)RS) and fluorescence activity,
whereby said at least one target-specific capture probe is either covalently bound to a SE(R)RS surface or said step (a) further comprising contacting said at least one target-specific capture probe with a SE(R)RS surface; and
(b) detecting the signal generated by said label in at least one surrogate hybrid formed by the binding of said at least one displaceable surrogate target probe and said at least one target-specific capture probe, which signal is proportionate to the presence and/or quantity of said at least one analyte in said sample.

2. The method of claim 1, wherein step (a) comprises the steps of:
(I) contacting said at least one target-specific capture probe with said at least one displaceable surrogate target probe so as to obtain at least one surrogate hybrid,
whereby said at least one target-specific capture probe is either covalently bound to a SE(R)RS surface and at least one surface-adsorbed surrogate hybrid is so obtained or said step (I) further comprises contacting said at least one target-specific capture probe with a SE(R)RS surface or said step (I) further comprises contacting said at least one surrogate hybrid with said SE(R)RS surface such that said at least one surrogate hybrid becomes adsorbed on said surface as at least one surface-adsorbed surrogate hybrid;
(II) contacting said at least one surface-adsorbed surrogate hybrid with said sample so as to allow displacement of said at least one displaceable surrogate target probe by said at least one analyte;
and wherein step (b) comprises the steps of:
(III) detecting the signal of said label in said at least one surface-adsorbed surrogate hybrid after step (I) using either or both SE(R)RS and fluorescence; and
(IV) detecting the signal of said at least one surface-adsorbed surrogate hybrid after step (II) using the same detection method or methods used in step (III);
wherein the difference in the signal obtained in steps (III) and (IV) is taken as proportionate to the presence and/or quantity of said at least one analyte in said sample.

3. The method according to claim 2 wherein step (a) comprises the steps of:
(V) contacting said at least one target-specific capture probe with said at least one displaceable surrogate target probe so as to obtain at least one surrogate hybrid;
(VI) contacting said at least one surrogate hybrid with said SE(R)RS surface such that said at least one surrogate hybrid becomes adsorbed on said surface forming at least one surface-adsorbed surrogate hybrid; and
(VII) contacting said at least one surface-adsorbed surrogate hybrid with said sample so as to allow displacement of said at least one displaceable surrogate target probe by said at least one analyte.

4. The method according to claim 2 wherein step II comprises ensuring appropriate annealing conditions to allow the hybridization of said at least one analyte in said sample with said at least one capture probe.

5. The method according to claim 1 wherein said at least one displaceable surrogate target probe comprises an oligonucleotide sequence which is not 100% complementary to said oligonucleotide sequence of said at least one capture probe.

6. The method according to claim 1, wherein said at least one target-specific capture probe further comprises a surface-seeking group and said at least one target-specific capture probe binds to said SE(R)RS surface through said surface-seeking group.

7. The method according to claim 1, which further comprises the step of
(c) contacting:
(iii) a standard capture probe comprising:
an oligonucleotide, and
a surface-seeking group, and
(iv) a standard probe comprising:
an oligonucleotide which is 100% complementary to the sequence of said standard capture probe,
a label which has either or both surface-enhanced (resonance) Raman scattering (SE(R)RS) and fluorescence activity,
thereby obtaining a standard hybrid, and
(v) a SE(R)RS surface;
thereby obtaining a surface-adsorbed standard hybrid; and
(d) detecting the signal generated by said label in said surface-adsorbed standard hybrid.

8. The method according to claim 7, wherein said standard hybrid obtained in step (c) is added to said sample in step (a) and wherein said standard probe comprises a label which is different from the label provided on said at least one surrogate target probe.

9. The method of claim 7, wherein said standard capture probe comprises an oligonucleotide sequence which does not hybridize with said target sequence.

10. The method according to claim 7, wherein said steps (c) and (d) are performed in a different vial than steps (a) and (b).

11. The method according to claim 10, wherein said sample in steps (a) and (b) is a control sample.

12. The method according to claim 1, wherein said at least one capture probe comprises a surface-seeking group.

13. The method according to claim 12, wherein said surface-seeking group is a benzotriazole.

14. The method according to claim 1, wherein said SE(R)RS surface consists of gold nanoparticles that are coated with silver by addition of silver hydroquinone after covalently linking said capture probe to said gold nanoparticles.

15. The method according to claim 1, wherein said SE(R)RS surface is a colloidal suspension of silver or gold nanoparticles, or aggregated colloids thereof

16. The method according to claim 1, wherein said SE(R)RS surface consists of silver or gold nanoparticles coated with a thin layer (1-few nanometers) of gold or silver, respectively.

17. The method according to claim 1, wherein said SE(R)RS surface consists of stable clusters of silver or gold nanoparticles.

18. The method according to claim 17, wherein said clusters are established by cross-linking with bi- or multifunctional macromolecules (telemers) which can bind chemically to said clusters.

19. A combination of surrogate target probe and capture probe comprising:
(i) a capture probe comprising:
an oligonucleotide,
(ii) a surrogate target probe comprising:
an oligonucleotide capable of binding to said capture probe,
whereby said oligonucleotide is characterized by a melting temperature that is lower than the melting temperature of an oligonucleotide that is 100% complementary to said capture probe's oligonucleotide,
a label attached to said surrogate target probe which has either or both surface-enhanced (resonance) Raman scattering (SE(R)RS) and fluorescence activity.

20. The combination of claim 19, wherein said capture probe is covalently bound to a SE(R)RS surface.

21. The combination of claim 19, wherein said capture probe comprises a surface-seeking group capable of binding a SE(R)RS surface.

22. A disposable cartridge (111) for use in a system for detecting the presence and/or quantity of at least one analyte in a sample, comprising
(a) a set of sources comprising:
source (101) of sample,
at least one source (102) of surrogate target,
at least one source (103) of target-specific capture probe,
at least one source (105) of additives serving in the detection;
(b) means (107) for contacting specified volumes from said sources; and
(c) means (106) for ensuring the provision of the fluids from said sources to the contacting means (107).

23. The cartridge of claim 22, which further comprises at least one source (104) of internal standard reagents.

24. The cartridge of claim 22, further comprising a window for detecting the signal generated at said contacting means.

25. A system (100) for detecting the presence or amount of at least one analyte in a sample comprising:
(a) means (107) for contacting said sample with:
(i) at least one target-specific capture probe comprising:
an oligonucleotide capable of specifically binding a target sequence within said analyte, and
(ii) at least one displaceable surrogate target probe comprising:
an oligonucleotide capable of specifically binding said capture probe,
a label which has either or both surface-enhanced (resonance) Raman scattering (SE(R)RS) and fluorescence activity; and
(b) means (108) for detecting the signal generated by said label in at least one surrogate hybrid formed by the binding of said at least one displaceable surrogate target probe and said at least one target-specific capture probe.

26. The system of claim 25, which further comprises a means for calculating (109) the amount of said at least one analyte by comparing the detection signals detected in said contacting means (107) at different time points in the provision of reagents from said sources to said contacting means (107).

27. The system of claim 25, wherein said means (108) for detecting comprises a light source, a filter and a detection means.
